(19) United States
(12) Patent Application Publication
(10) Pub. No.: US 2020/0229719 A1
(43) Pub. Date: Jul. 23, 2020

(54) PHYSIOLOGICAL SENSING METHOD AND DEVICE USING THE SAME

(71) Applicant: NATIONAL CHIAO TUNG UNIVERSITY, HSINCHU CITY (TW)

(72) Inventors: CHEN-YI LEE, HSINCHU CITY (TW); EUGENE LEE, HSINCHU CITY (TW); TSU JUI HSU, CHANGHUA COUNTY (TW)

(21) Appl. No.: 16/441,801

(22) Filed: Jun. 14, 2019

(30) Foreign Application Priority Data
Jan. 18, 2019 (TW) 108102090

(51) Int. Cl. A61B 5/024; A61B 5/00

(52) U.S. Cl. CPC A61B 5/02416; A61B 5/1455; A61B 5/0095; A61B 5/681

(57) ABSTRACT

A physiological sensing method and a device using the same is disclosed. The two sides of the device are respectively provided with an installed component. The device is arranged on the wrist of a user through the installed components. At least two physiological sensors, arranged in row on the installed component, generate and transmit original physiological signals to a controller. The controller filters out the original physiological signals to generate two basic energy values, multiplies the two basic physiological parameters by the two basic energy values to generate two basic estimation parameters, and substitutes the two basic energy values into a standard physiological value equation to generate a standard physiological value.

This application claims priority for Taiwan patent application no. 108102090 filed on Jan. 18, 2019, the content of which is incorporated by reference in its entirely.

### BACKGROUND OF THE INVENTION

### Field of the Invention

The present invention relates to physiological detecting technology, particularly to a physiological sensing method and a device using the same.

### Description of the Related Art

Photoplethysmography (PPG) signals are obtained from changes of peripheral blood circulation of a human body. That is to say, PPG signals are obtained from changes of blood in human blood vessels.

At present, most PPG signals are obtained by the pulse oximter. The sensor of the pulse oximeter is fixed to the skin of the finger of a user through a fixture. The lighting element of the sensor projects an optical signal on the skin of the finger of the user. Since the blood flow per unit area of the blood vessels on the skin changes with the pulsation of the heart, the optical signal reflected by the skin changes as the blood changes. The sensor obtains the PPG signal according to the variation of the reflected optical signal. The pulse oximeter calculates physiological signals for blood sugar, heart rate, heart rate variability (HRV), blood oxygen, or blood pressure according to various parameters of the PPG signal. Besides, the PPG signal is also used to monitor respiration, hypovolemia, or other circulation situations.

Since the PPG signal is generated by retrieving the variation of the optical signal reflected by the skin, the sensor obtains the PPG signal without invading the human body. The PPG signal itself has abundant physiological information, which is very helpful in the medical inspection technology. As a result, many research institutes have invested in the research of PPG signals.

However, the conventional technology retrieves PPG signals through the blood flow of the micro-vessels of the finger of the skin. Thus, when the user is measured, the finger is clamped by the fixture of the pulse oximeter. The user can not simultaneously handle other things, which makes the user inconveniently move. Accordingly, the user cannot wear the fixture to measure the PPG signal for a long time. In addition, the conventional pulse oximeter uses a single sensor to measure. When the only sensor obtains the signal, the signal distortion is easily caused due to the interference of the outside or the unstable interior of the pulse oximeter. Therefore, there are many uncertainties for retrieving PPG signals, which is a major researching focus that needs to be overcome.

To overcome the abovementioned problems, the present invention provides a physiological sensing method and a device using the same, so as to solve the afore-mentioned problems of the prior art.

### SUMMARY OF THE INVENTION

The primary objective of the present invention is to provide a physiological sensing method and a device using the same, which uses a way of estimating physiological signals to provide a higher ratio for a sensor with higher precision, thereby estimating a physiological signal with high precision.

Another objective of the present invention is to provide a physiological sensing method and a device using the same, wherein the device is worn by the wrist of a user to directly sense the blood flow of the artery of a user. The physiological sensors are arranged into an array fitting wrists of different users, thereby improving the precision of measuring physiological signals.

Further objective of the present invention is to provide a physiological sensing method and a device using the same, wherein the device is worn comfortably and helpful in detecting the blood flow of the artery of a user for a long time.

To achieve the abovementioned objectives, the present invention provides a physiological sensing method comprising: inputting at least two original physiological signals and filtering out the at least two original physiological signals to generate at least two basic physiological parameters; substituting the two basic physiological parameters into a basic energy equation to convert the two basic physiological parameters, thereby generating two basic energy values; multiplying the two basic physiological parameters by the two basic energy values to generate two basic estimation parameters; and substituting the two basic energy values, the two basic estimation parameters, an original reference energy value, and a reference estimation parameter into a standard physiological value equation to generate a standard physiological value.

In an embodiment of the present invention, the physiological sensing method further comprises a step of substituting the two basic energy values into a conversion equation to generate a new reference energy value and replacing the original reference energy value with the new reference energy value, after the step of replacing the original reference energy value with the new reference energy value, returning to the step of inputting the at least two original physiological signals, and the conversion equation is expressed as follows:

$${b}_{c}^{+} ( t ) = {\sum }_{i = 1}^{N} ( \frac{{b}_{c}^{-}}{N} + {b}_{i} ) ; and$$

$${b}_{c}^{-} ( t + 1 ) = {( {{b}_{c}^{+} ( t )}^{- 1} + {‖ Q ‖}_{2}^{2} )}^{- 1} ,$$

wherein b_{c}⁺(t) represents the new reference energy value, b_{c}⁻ represents the original reference energy value, bᵢ represents the basic energy value, and Q represents a covariance of process noise.

In an embodiment of the present invention, the basic physiological signal is expressed by follows:

\hat{x}ᵢ=(zᵢ(t:t+τ)⊙w(t))*h(t),

wherein \hat{x}ᵢ represents the basic physiological signal, zᵢ represents the original physiological signal, t represents the time instance of the original physiological signal, τ represents a size of an observation window, w(t) represents the Hamming window function, and h(t) represents the finite impulse response (FIR) function.

In an embodiment of the present invention, the basic energy equation is expressed by follows:

bᵢ=∥\hat{x}ᵢ∥_{\hat{x}}²,

wherein bᵢ represents the basic energy value and \hat{x}ᵢ represents the basic physiological signal.

In an embodiment of the present invention, the basic estimation parameter is expressed by follows:

vᵢ=bᵢ\hat{x}ᵢ,

wherein vᵢ represents the basic estimation parameter, bᵢ represents the basic energy value, and \hat{x}ᵢ represents the basic physiological signal.

In an embodiment of the present invention, the standard physiological value equation is expressed as follows:

$${x}_{c}^{+} ( t ) = {( {\sum }_{i = 1}^{N} ( \frac{{b}_{c}^{-}}{N} + {b}_{i} ) )}^{- 1} {\sum }_{i = 1}^{N} ( \frac{{v}_{c}^{-}}{N} + {v}_{i} ) ,$$

wherein x_{c}⁺(t) represents the standard physiological value, N represents the number of sensors inputting the at least two original physiological signals, b_{c}⁻ represents the original reference energy value, bᵢ represents the basic energy value, vᵢ represents the basic estimation parameter, and v_{c}⁻ represents the reference estimation parameter.

The present invention provides a physiological sensing device with two sides thereof respectively provided with two installed components, the physiological sensing device arranged on a wrist of a user through the installed components, and the physiological sensing device comprising: at least two physiological sensors, arranged in row on at least one of the installed components and an artery of the user, respectively generating at least two original physiological signals corresponding to the artery; a database storing at least one original reference energy value, at least one reference estimation parameter, a basic energy equation, and a standard physiological value equation; a controller, signally connected to the at least two physiological sensors and the database, receiving the at least two original physiological signals, filtering out the at least two original physiological signals to generate at least two basic physiological parameters, substituting the two basic physiological parameters into the basic energy equation to generate two basic energy values, multiplying the two basic physiological parameters by the two basic energy values to generate two basic estimation parameters, retrieving the at least one original reference energy value, the at least one reference estimation parameter, and the standard physiological value equation, and substituting the two basic energy values, the two basic estimation parameters, the original reference energy value, and the reference estimation parameter into the standard physiological value equation to generate a standard physiological value; and a display, electrically connected to the controller, receiving and displaying the standard physiological value.

In an embodiment of the present invention, the physiological sensing device further comprises an accelerometer signally connected to the controller, the accelerometer generates and transmits an acceleration value to the controller, and the controller ignores the at least two original physiological signals transmitted at present when the controller determines that the acceleration value is larger than a given value.

Below, the embodiments are described in detail in cooperation with the drawings to make easily understood the technical contents, characteristics and accomplishments of the present invention.

### BRIEF DESCRIPTION OF THE DRAWINGS

FIG. 1 is a perspective view of a physiological sensing device according to an embodiment of the present invention;

FIG. 2 is a perspective view of wearing a physiological sensing device according to an embodiment of the present invention;

FIG. 3 is a diagram schematically showing a physiological sensing device according to an embodiment of the present invention;

FIG. 4 is a flowchart of a physiological sensing method according to an embodiment of the present invention; and

FIG. 5 is a diagram schematically showing waveforms of signals according to an embodiment of the present invention.

### DETAILED DESCRIPTION OF THE INVENTION

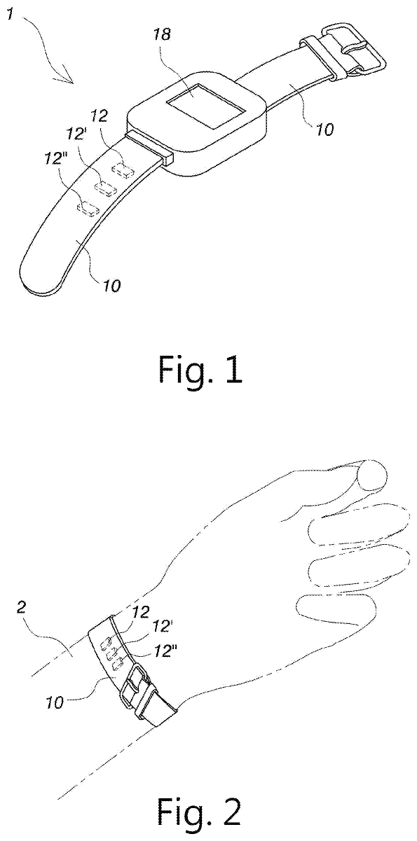

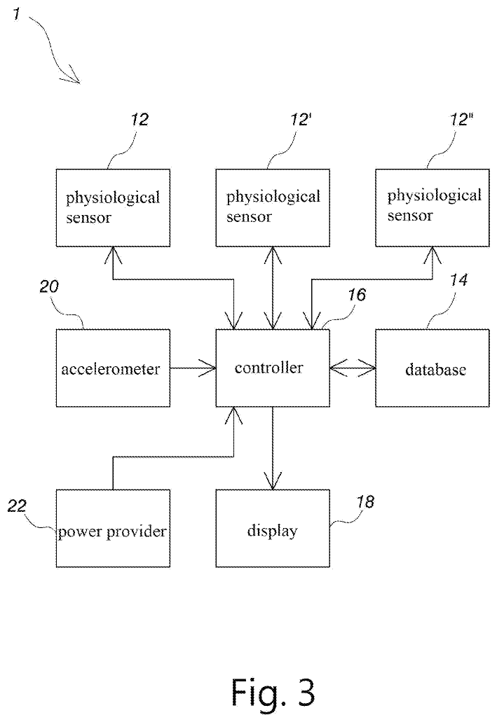

Refer to FIG. 1, FIG. 2, and FIG. 3. The structure of the present invention is described as follows. The two sides of the physiological sensing device 1 of the present invention are respectively provided with two installed components 10. For example, the installed component 10 may be a strap. The physiological sensing device 1, arranged on the wrist 2 of a user through the installed components 10, measures the blood flow of the artery of the wrist 2 of the user. In the embodiment, the physiological sensing device 1 comprises three physiological sensors 12, 12′, and 12″, a database 14, a controller 16, a display 18, an accelerometer 20, and a power provider 22. The physiological sensors 12, 12′, and 12″ are arranged in row on at least one of the installed components 10 and an artery of the wrist 2 of the user when the installed component 10 is tied to the user. The physiological sensors 12, 12′, and 12″ measure the blood flow of the artery. The physiological sensors 12, 12′, and 12″ respectively generate at least three original physiological signals corresponding to the artery. The database 14 stores at least one original reference energy value, at least one reference estimation parameter, a basic energy equation, and a standard physiological value equation.

Refer to FIG. 1, FIG. 2, and FIG. 3. The controller 16 is signally connected to the physiological sensors 12, 12′, and 12″, the database 14, the display 18, the accelerometer 20, and the power provider 22. The controller 16 receives the three original physiological signals generated by the physiological sensors 12, 12′, and 12″. The original physiological signals may be photoplethysmography (PPG) signals being analog signals. The controller 16 converts the original physiological signals into digital signals. The controller 16 retrieves the data stored in the database 14 and uses the data to process the original physiological signals, thereby generating a standard physiological value. The display 18 receives and displays the standard physiological value. In addition, the controller 16 determines physiological signals for blood sugar, heart rate, heart rate variability (HRV), blood oxygen, or blood pressure according to the PPG signals.

The accelerometer 20 generates and transmits an acceleration value to the controller 16. The accelerometer 20 detects the present position and generates and transmits values X, Y, and Z in the X, Y, and Z axes to the controller 16. The controller 16 substitutes values X, Y, and Z into a conversion equation (1) stored in the database 14 to generate X_{acc}, Y_{acc}, and Z_{acc}. The conversion equation (1) is expressed as follows:

X_{acc}=X(t:t+τ)_{acc}

Y_{acc}=Y(t:t+τ)_{acc}

Z_{acc}=Z(t:t+τ)_{acc} (1)

Wherein, X is the value of the present position in the X axis, Y is the value of the present position in the Y axis, Z is the value of the present position in the Z axis, r represents the size of an observation window, and t represents time. Then, the controller 16 substitutes X_{acc}, Y_{acc}, and Z_{acc} into an acceleration determining equation (2).

[symbol]

[|X_{acc}−\hat{X}_{acc}|²]+ [symbol] [|Y_{acc}−Ŷ_{acc}|²]+ [symbol] [|Z_{acc}−\hat{Z}_{acc}|²]<ε (2)

Wherein, \hat{⋅} represents a mean operator, [symbol] [⋅] represents an expectation operator, ε represents a given value. In the embodiment, the size of the observation window is 2 seconds. The number of samples depends on the sampling frequency of the accelerometer 20. From the acceleration determining equation (2), it is known that the controller 16 ignores the three original physiological signals presently transmitted by the physiological sensors 12, 12′, and 12″ when the controller 16 determines that the acceleration value is larger than the given value ε, such as 5. The mechanism can ignore the original physiological signals that cause the signal distortion due to the great shake of the user, so as to improve the precision of the signal. The power provider 22 may be a battery that provides power for the controller 16 and drives the controller 16. The controller 16 transmits power to the physiological sensors 12, 12′, and 12″, the database 14, the display 18, and the accelerometer 20, which are signally connected to the controller 16, and drives the physiological sensors 12, 12′, and 12″, the database 14, the display 18, and the accelerometer 20.

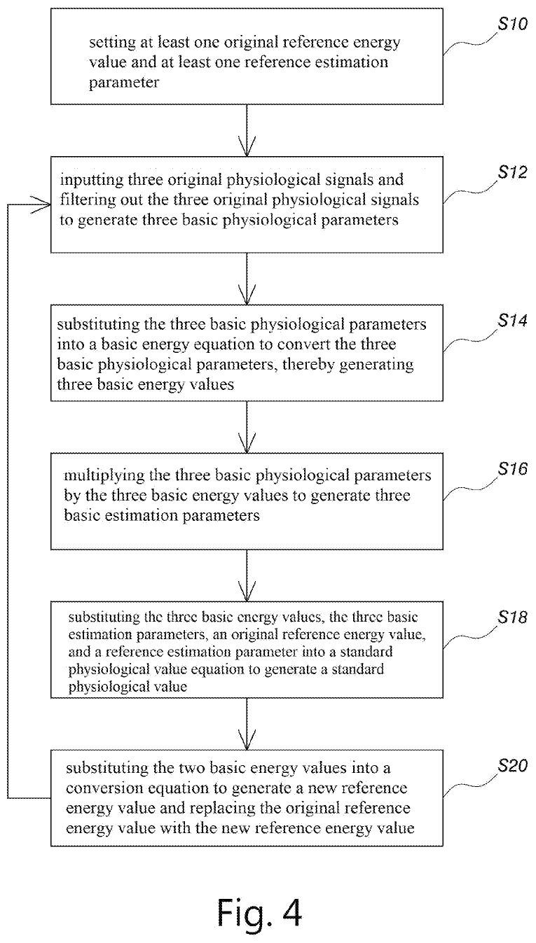

After describing the structure of the physiological sensing device 1, the physiological sensing method using the physiological sensing device 1 is described in detail. Refer to FIG. 1, FIG. 2, FIG. 3, and FIG. 4. Firstly, in Step S10, at least one original reference energy value and at least one reference estimation parameter are set in the database 14. Then, in Step S12, the three physiological sensors 12, 12′, and 12″ sense different positions of the wrist 2 of the user and input the three original physiological signals to the controller 16. The controller 16 uses the value generated by the accelerometer 20 to determine whether the original physiological signals transmitted at present are ignored. If the answer is yes, nothing is performed on the three original physiological signals. If the three original physiological signals are used, the controller 16 uses the Hamming window function and the finite impulse response (FIR) function to filter out the original physiological signals z₁, z₂, and z₃ generated by the physiological sensors 12, 12′, and 12″, thereby generating three basic physiological parameters \hat{x}₁, \hat{x}₂, and \hat{x}₃. The basic physiological signal is expressed as follows:

\hat{x}ᵢ=(zᵢ(t:t+τ)⊙w(t))*h(t) (3)

Wherein, \hat{x}ᵢ represents the basic physiological signal, zᵢ represents the original physiological signal, t represents a time instance of the original physiological signal, τ represents the size of the observation window, namely a signal length of the detected original physiological signal, w(t) represents the Hamming window function, and h(t) represents the finite impulse response (FIR) function. In addition, the controller 16 determines physiological signals for blood sugar, heart rate, heart rate variability (HRV), blood oxygen, or blood pressure according to the original physiological signals.

Then, in Step S14, the controller 16 retrieves a basic energy equation from the database 14 and substitutes the three basic physiological parameters \hat{x}₁, \hat{x}₂, and \hat{x}₃ in Step S12 into the basic energy equation to generate three basic energy values b₁, b₂, and b₃. The basic energy equation is expressed as follows:

bᵢ=∥\hat{x}ᵢ∥₂² (4)

Wherein, bᵢ represents the basic energy value and \hat{x}ᵢ represents the basic physiological signal. After converting the basic physiological parameters \hat{x}₁, \hat{x}₂, and \hat{x}₃ into the basic energy values b₁, b₂, and b₃, the process proceeds to Step S16. The controller 16 multiplies the three basic physiological parameters \hat{x}₁, \hat{x}₂, and \hat{x}₃ by the three basic energy values b₁, b₂, and b₃ to generate three basic estimation parameters. The basic estimation parameter is expressed by follows:

vᵢ=bᵢ\hat{x}ᵢ (5)

Wherein, vᵢ represents the basic estimation parameter, bᵢ represents the basic energy value, and \hat{x}ᵢ represents the basic physiological signal. Specifically, the three basic physiological parameters \hat{x}₁, \hat{x}₂, and \hat{x}₃ multiplied by the three basic energy values b₁, b₂, and b₃ is expressed as follows:

v₁=b₁\hat{x}₁

v₂=b₂\hat{x}₂

v₃=b₃\hat{x}₃

Then, in Step S18, the controller 16 substitutes the basic energy values b₁, b₂, and b₃ in Step S14, the basic estimation parameters v₁, v₂, and v₃ in Step S16, and the original reference energy value b_{c}⁻ and the reference estimation parameter v_{c}⁻ into the standard physiological value equation retrieved from the database 14 to generate the standard physiological value. The standard physiological value equation is expressed as follows:

$${x}_{c}^{+} ( t ) = {( {\sum }_{i = 1}^{N} ( \frac{{b}_{c}^{-}}{N} + {b}_{i} ) )}^{- 1} {\sum }_{i = 1}^{N} ( \frac{{v}_{c}^{-}}{N} + {v}_{i} ) \qquad ( 6 )$$

Wherein, xₓ⁺(t) represents the standard physiological value, N represents the number of sensors inputting the original physiological signals, b_{c}⁻ represents the original reference energy value, bᵢ represents the basic energy value, vᵢ represents the basic estimation parameter, and v_{c}⁻ represents the reference estimation parameter. After generating the standard physiological value, the controller 16 transmits the standard physiological value to the display 18. The display receives and displays the standard physiological value provided to the user for reference.

After calculating the standard physiological value, the process proceeds to Step S20. Finally, in Step S20, the controller 16 substitutes the basic energy values b₁, b₂, and b₃ in Step S14 into a conversion equation to generate a new reference energy value and replaces the original reference energy value with the new reference energy value. Then, the process returns to Step S12 to input the three original physiological signals. The conversion equation is expressed as follows:

$${b}_{c}^{+} ( t ) = {\sum }_{i = 1}^{N} ( \frac{{b}_{c}^{-}}{N} + {b}_{i} ) \\ {b}_{c}^{-} ( t + 1 ) = {( {{b}_{c}^{+} ( t )}^{- 1} + {‖ Q ‖}_{2}^{2} )}^{- 1} \qquad ( 7 )$$

Wherein, bₓ⁺(t) represents the new reference energy value, b_{c}⁻ represents the original reference energy value, bᵢ represents the basic energy value, and Q represents a covariance of process noise. Q is used as a normalized term that controls the weight of the previous messages. On top of that, the controller 16 converts the standard physiological value in Step S18 into a reference physiological signal and stores the reference physiological signal in the database 14, wherein the reference physiological signal is referenced by subsequent signals. The reference physiological signal is expressed as follows:

x_{c}⁻(t+1)=xₓ⁺(t) (8)

Wherein, x_{c}⁻(t+1) represents the reference physiological signal and x_{c}⁺(t) represents the standard physiological value.

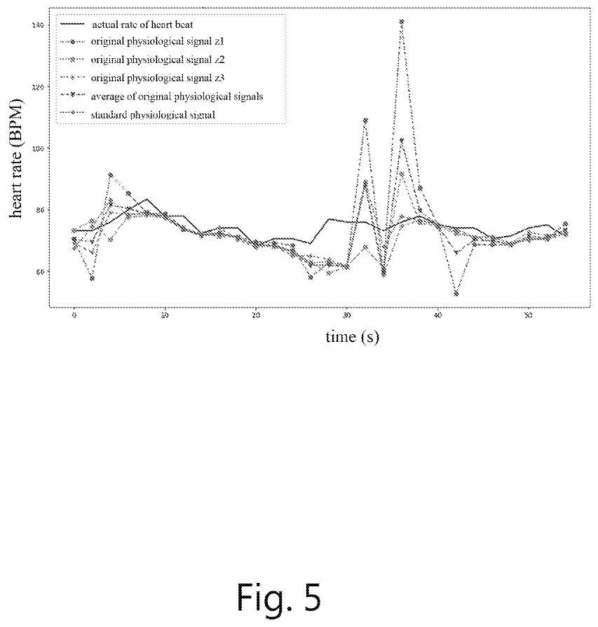

Refer to FIG. 5. FIG. 5 is a diagram schematically showing experiment data, which represent the actual rate of heart beat, the three original physiological signals z₁, z₂, and z₃ sensed by the physiological sensors 12, 12′, and 12″, the average of the three original physiological signals z₁, z₂, and z₃, and the standard physiological value during a time interval. From FIG. 5, it can be seen that the original physiological signal z₁ greatly varies from the 30^{th} second to the 40^{th} second. Thus, it is determined that the physiological sensor 12 generates the distorted original physiological signal z₁ due to the great shake of the user from the 30^{th} second to the 40^{th} second. As a result, the average of the three original physiological signals z₁, z₂, and z₃ greatly varies from the 30^{th} second to the 40^{th} second due to the great variation of the original physiological signal z₁. On the contrary, the present invention filters out the distorted signals generated due to the great shake of the user according to the determination of the accelerometer 20. Besides, the present invention assigns a higher weight to the more stable signal. Compared with the average of the original physiological signals, the standard physiological signal is more stable. The standard physiological signal is difficultly distorted when the original physiological signal is distorted.

The reason of generating the more stable standard physiological value is that the basic energy equation (4) makes the more stable basic physiological signal \hat{x}ᵢ generate the higher basic energy value bᵢ. The higher the basic energy value bᵢ, the higher the basic estimation parameter vᵢ generated according to the basic energy value bᵢ. The more stable signal has the higher weight when the basic energy value bᵢ and the basic estimation parameter vᵢ are substituted into the standard physiological value equation (6). Therefore, the present invention can generate the precise standard physiological value.

In conclusion, the present invention uses a special way for estimating physiological signals to provide a higher ratio for a sensor with higher precision, thereby estimating a physiological signal with high precision. The physiological sensors of the physiological sensing device of the present invention, arranged into an array fitting wrists of different users, sense the blood flow of the artery of the wrist of the user, thereby improving the precision of measuring physiological signals. Additionally, the physiological sensing device is worn comfortably and helpful in detecting the blood flow of the artery of a user for a long time.

The embodiments described above are only to exemplify the present invention but not to limit the scope of the present invention. Therefore, any equivalent modification or variation according to the shapes, structures, features, or spirit disclosed by the present invention is to be also included within the scope of the present invention.

What is claimed is:

1. A physiological sensing method comprising:
inputting at least two original physiological signals and filtering out the at least two original physiological signals to generate at least two basic physiological parameters;
substituting the two basic physiological parameters into a basic energy equation to convert the two basic physiological parameters, thereby generating two basic energy values;
multiplying the two basic physiological parameters by the two basic energy values to generate two basic estimation parameters; and
substituting the two basic energy values, the two basic estimation parameters, an original reference energy value, and a reference estimation parameter into a standard physiological value equation to generate a standard physiological value.

2. The physiological sensing method according to claim 1, further comprising a step of substituting the two basic energy values into a conversion equation to generate a new reference energy value and replacing the original reference energy value with the new reference energy value, after the step of replacing the original reference energy value with the new reference energy value, returning to the step of inputting the at least two original physiological signals, and the conversion equation is expressed as follows:
$${b}_{c}^{+} ( t ) = {\sum }_{i = 1}^{N} ( {b}_{c}^{-} + {b}_{i} ) ; and$$
$${b}_{c}^{-} ( t + 1 ) = {( {{b}_{c}^{+} ( t )}^{- 1} + {‖ Q ‖}_{2}^{2} )}^{- 1} ,$$
wherein b_{c}⁺(t) represents the new reference energy value, b_{c}⁻ represents the original reference energy value, bᵢ represents the basic energy value, and Q represents a covariance of process noise.

3. The physiological sensing method according to claim 1, wherein the basic physiological signal is expressed by follows:
\hat{x}ᵢ=(zᵢ(t:t+τ)⊙w(t))*h(t),
wherein \hat{x}ᵢ represents the basic physiological signal, zᵢ represents the original physiological signal, t represents a time instance of the original physiological signal, τ represents a size of an observation window, w(t) represents a Hamming window function, and h(t) represents a finite impulse response (FIR) function.

4. The physiological sensing method according to claim 1, wherein the basic energy equation is expressed by follows:
bᵢ=∥xᵢ∥₂²,
wherein bᵢ represents the basic energy value and \hat{x}ᵢ represents the basic physiological signal.

5. The physiological sensing method according to claim 1, wherein the basic estimation parameter is expressed by follows:
vᵢ=bᵢ\hat{x}ᵢ,
wherein vᵢ represents the basic estimation parameter, bᵢ represents the basic energy value, and \hat{x}ᵢ represents the basic physiological signal.

6. The physiological sensing method according to claim 1, wherein the standard physiological value equation is expressed as follows:
$${x}_{c}^{+} ( t ) = {( {\sum }_{i = 1}^{N} ( \frac{{b}_{c}^{-}}{N} + {b}_{i} ) )}^{- 1} {\sum }_{i = 1}^{N} ( \frac{{v}_{c}^{-}}{N} + {v}_{i} ) ,$$
wherein xₓ⁺(t) represents the standard physiological value, N represents number of sensors inputting the at least two original physiological signals, b_{c}⁻ represents the original reference energy value, bᵢ represents the basic energy value, vᵢ represents the basic estimation parameter, and v_{c}⁻ represents the reference estimation parameter.

7. The physiological sensing method according to claim 1, wherein in the step of inputting the at least two original physiological signals, the at least two original physiological signals are inputted and physiological signals for blood sugar, heart rate, heart rate variability (HRV), blood oxygen, or blood pressure are determined according to the original physiological signals.

8. The physiological sensing method according to claim 1, wherein the original reference energy value and the reference estimation parameter are set before inputting the at least two original physiological signals.

9. A physiological sensing device with two sides thereof respectively provided with two installed components, the physiological sensing device arranged on a wrist of a user through the installed components, and the physiological sensing device comprising:
at least two physiological sensors, arranged in row on at least one of the installed components and an artery of the user, respectively generating at least two original physiological signals corresponding to the artery;
a database storing at least one original reference energy value, at least one reference estimation parameter, a basic energy equation, and a standard physiological value equation;
a controller, signally connected to the at least two physiological sensors and the database, receiving the at least two original physiological signals, filtering out the at least two original physiological signals to generate at least two basic physiological parameters, substituting the two basic physiological parameters into the basic energy equation to generate two basic energy values, multiplying the two basic physiological parameters by the two basic energy values to generate two basic estimation parameters, retrieving the at least one original reference energy value, the at least one reference estimation parameter, and the standard physiological value equation, and substituting the two basic energy values, the two basic estimation parameters, the original reference energy value, and the reference estimation parameter into the standard physiological value equation to generate a standard physiological value; and
a display, electrically connected to the controller, receiving and displaying the standard physiological value.

10. The physiological sensing device according to claim 9, further comprising an accelerometer signally connected to the controller, the accelerometer generates and transmits an acceleration value to the controller, and the controller ignores the at least two original physiological signals transmitted at present when the controller determines that the acceleration value is larger than a given value.

11. The physiological sensing device according to claim 9, further comprising a power provider electrically connected to the controller, and the power provider provides power for the controller.

12. The physiological sensing device according to claim 9, wherein the database further stores a conversion equation, the controller substitutes the two basic energy values into the conversion equation to generate a new reference energy value and replacing the original reference energy value with the new reference energy value, returning to the step of inputting the at least two original physiological signals after replacing the original reference energy value with the new reference energy value, and the conversion equation is expressed as follows:
$${b}_{c}^{+} ( t ) = {\sum }_{i = 1}^{N} ( \frac{{b}_{c}^{-}}{N} + {b}_{i} ) ; and$$
$${b}_{c}^{-} ( t + 1 ) = {( {{b}_{c}^{+} ( t )}^{- 1} + {‖ Q ‖}_{2}^{2} )}^{- 1} ,$$
wherein bₓ⁺(t) represents the new reference energy value, bₓ⁻ represents the original reference energy value, bᵢ represents the basic energy value, and Q represents a covariance of process noise.

13. The physiological sensing device according to claim 9, wherein the basic physiological signal is expressed by follows:
\hat{x}ᵢ=(zᵢ(t:t+τ)⊙w(t))*h(t),
wherein \hat{x}ᵢ represents the basic physiological signal, zᵢ represents the original physiological signal, t represents a time instance of the original physiological signal, τ represents a size of an observation window, w(t) represents a Hamming window function, and h(t) represents a finite impulse response (FIR) function.

14. The physiological sensing device according to claim 9, wherein the basic energy equation is expressed by follows:
bᵢ=∥xᵢ∥₂²,
wherein bᵢ represents the basic energy value and \hat{x}ᵢ represents the basic physiological signal.

15. The physiological sensing device according to claim 9, wherein the basic estimation parameter is expressed by follows:
vᵢ=bᵢ\hat{x}ᵢ,
wherein vᵢ represents the basic estimation parameter, bᵢ represents the basic energy value, and \hat{x}ᵢ represents the basic physiological signal.

16. The physiological sensing device according to claim 9, wherein the standard physiological value equation is expressed as follows:
$${x}_{c}^{+} ( t ) = {( {\sum }_{i = 1}^{N} ( \frac{{b}_{c}^{-}}{N} + {b}_{i} ) )}^{- 1} {\sum }_{i = 1}^{N} ( \frac{{v}_{c}^{-}}{N} + {v}_{i} ) ,$$
wherein xₓ⁺(t) represents the standard physiological value, N represents number of sensors inputting the at least two original physiological signals, b_{c}⁻ represents the original reference energy value, bᵢ represents the basic energy value, vᵢ represents the basic estimation parameter, and v_{c}⁻ represents the reference estimation parameter.

17. The physiological sensing device according to claim 9, wherein the controller determines physiological signals for blood sugar, heart rate, heart rate variability (HRV), blood oxygen, or blood pressure according to the original physiological signals.

18. The physiological sensing device according to claim 9, wherein the installed component is a strap.
